(19) United States
(12) Patent Application Publication
(10) Pub. No.: US 2021/0169814 A1
(43) Pub. Date: Jun. 10, 2021

(54) COMPOSITIONS AND METHODS FOR MASKING THE TASTE OF DRUGS

(71) Applicant: UNM RAINFOREST INNOVATIONS, Albuquerque, NM (US)

(72) Inventors: Linda A. Felton, Albuquerque, NM (US); Pavan Muttil, Albuquerque, NM (US)

(21) Appl. No.: 17/111,645

(22) Filed: Dec. 4, 2020

Related U.S. Application Data
(60) Provisional application No. 62/943,652, filed on Dec. 4, 2019.

(51) Int. Cl. A61K 9/50

(52) U.S. Cl. CPC A61K 9/5073; A61K 9/5026; A61K 9/5089; A61K 9/5047; A61K 9/5036

(57) ABSTRACT

A pharmaceutical composition generally includes a primary microparticle having at least one drug dispersed within a matrix and a taste masking seal coating dispersed over at least a portion of the primary microparticle. In some embodiments, the seal coating can include a reverse enteric coating. In other embodiments, the seal coating can include a water-soluble polymer such as carrageenan, hypromellose, or polyvinyl alcohol. In some embodiments, the seal coating is continuous. In other embodiments, the seal coating is discontinuous. The pharmaceutical composition can be prepared using a two-step spray drying process.

### CROSS-REFERENCE TO RELATED APPLICATION

This application claims the benefit of U.S. Provisional Patent Application No. 62/943,652, filed Dec. 4, 2019, which is incorporated herein by reference in its entirety.

### GOVERNMENT FUNDING

This invention was made with government support under HD095100 awarded by the National Institutes of Health. The government has certain rights in the invention.

### SUMMARY

This disclosure describes, in one aspect, a pharmaceutical composition. Generally, the pharmaceutical composition includes a primary microparticle having at least one drug dispersed within a matrix and a taste masking seal coating dispersed over at least a portion of the primary microparticle.

In some embodiments, the seal coating can include a reverse enteric coating. In other embodiments, the seal coating can include a water-soluble polymer such as carrageenan, hypromellose, or polyvinyl alcohol.

In some embodiments, the primary microparticle can include two or more drugs dispersed within the matrix.

In another aspect, this disclosure describes a method for preparing a pharmaceutical composition. Generally, the method incudes obtaining a primary microparticle and applying a seal coating to at least a portion of the primary microparticle, the seal coating comprising a pharmaceutically acceptable polymer. The primary microparticle generally includes a matrix and drug dispersed within the matrix.

In some embodiments, the primary microparticle includes at least two drugs dispersed within the matrix.

In some embodiments, the seal coating includes a reverse enteric coating.

In some embodiments, the seal coating is continuous. In other embodiments, the seal coating is discontinuous.

In some embodiments, applying the seal coating to the particles can include suspending a plurality of the primary microparticles in a suitable solvent, dissolving the pharmaceutically acceptable polymer in an appropriate solvent, and spray drying the primary microparticles with the pharmaceutically acceptable polymer.

In some embodiments, applying the seal coating to the particles can include suspending a plurality of the primary microparticles in an aqueous medium, dissolving a reverse enteric polymer in an aqueous medium having a pH sufficiently acidic to dissolve the reverse enteric polymer and spray drying the primary microparticles with the dissolved reverse enteric polymer.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

The patent or application file contains at least one drawing executed in color. Copies of this patent or patent application publication with color drawings will be provided by the Office upon request and payment of the necessary fee.

FIG. 1A. Schematic illustration of microparticles. Matrix microparticles produced from conventional spray drying methods.

FIG. 1B. Schematic illustration of microparticles. Sealed matrix microparticles.

FIG. 2. Schematic illustration of a three-way spray nozzle.

FIG. 3A. Structure of repeat unit of EUDRAGIT E (Evonik Industries, Essen, Germany).

FIG. 3B. Structure of repeat unit of carrageenan.

FIG. 3C. Structure of repeat unit of KOLLICOAT Smartseal (BASF, Ludwigshafen, Germany).

FIG. 4A. Two-step spray drying process. Step 1 produces matrix microparticles.

FIG. 4B. Two-step spray drying process. Step 2 involves a three-way nozzle and produces sealed matrix microparticles.

FIG. 5. Fluorescent microscopic image of conventional spray dried microparticles. Green: acetaminophen.

FIG. 6. Fluorescent microscopic image of sealed microparticles. Red and green channels merged. Red: Rhodamine B in seal coating; Green: acetaminophen.

### DETAILED DESCRIPTION OF ILLUSTRATIVE EMBODIMENTS

This disclosure describes a micron-sized, taste masking drug delivery platform for a wide range of drugs that can be readily incorporated into a variety of oral dosage forms, including suspensions, orally dissolving tablets, other multiparticulate systems, and orodispersible films. Generally, drugs prepared using the taste masking delivery platform include micron-sized particles that may be easily swallowed and are sufficiently small to avoid grittiness in the oral cavity. The taste masking drug deliver platform is compatible with a wide range of drugs, irrespective of their solubility, ionic nature, or required dose.

In another aspect, this disclosure describes sealed matrix microparticles that are more palatable than conventional spray dried matrices.

In another aspect, this disclosure describes a two-step spray drying process for preparing taste-masked micron-sized particles. Spray drying is commonly used in the pharmaceutical industry to prepare microparticles for incorporation into various dosage forms. These particles, however, are matrices where drug is distributed throughout the particle, including on the surface of the particle, which can thus interact with taste buds when administered orally. Therefore, conventional spray drying does not effectively mask taste, especially for aggressively bitter drugs. In some embodiments, the process uses an aqueous-based, two-step spray drying process to seal conventional matrix microparticles with reverse enteric polymers to interfere with drug on the exterior surface of the particle from interacting with taste buds.

The taste masking drug delivery platform described herein can be used to mask the taste of any poorly palatable chemical entity, which could facilitate the drug development process, allowing more palatable products to reach patients more rapidly.

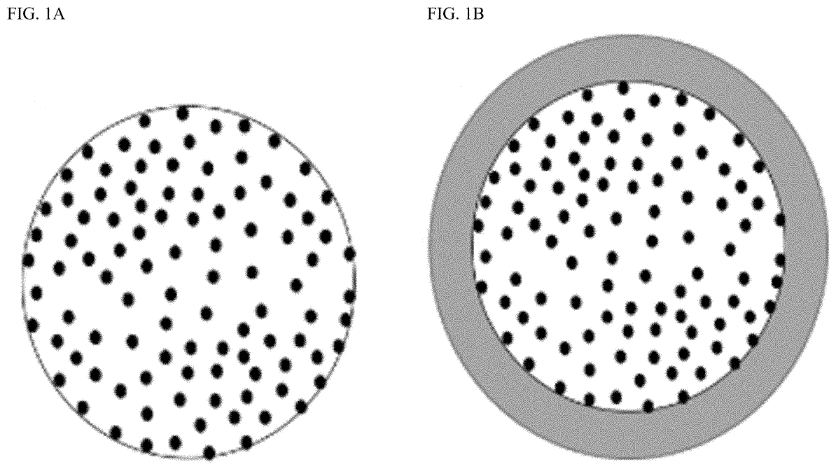

Conventional spray drying produces matrix microparticles illustrated schematically in FIG. 1A. The taste masking drug delivery platform described herein further seal the microparticles, as illustrated schematically in FIG. 1B. Spray drying involves at least one material being either dissolved or dispersed in a solvent that is atomized with air or an inert gas. The solvent evaporates as it exits the spray nozzle and thus forms solid micron-sized particles, typically in the range of 1 μm to about 25 μm in diameter, which are then collected in a vessel. Spray drying is commonly used in the pharmaceutical industry and can, to some extent, improve the taste of small doses (e.g., <10 mg) of certain drugs. Conventional spray drying, however, produces matrix microparticles in which drug is distributed throughout the matrix, including on the particle surface (FIG. 1A). Drug at the surface of the particle can interact with taste buds. Therefore, conventional spray drying cannot effectively mask taste, especially for aggressively bitter drugs.

To overcome this shortcoming, the taste masking drug delivery platform described herein provides a two-step spray drying process. The first step of the two-step process includes preparing a primary drug microparticle such as, for example, a microparticle prepared by conventional spray drying techniques. The primary drug microparticle can include a single drug or a combination of two or more drugs. In some embodiments, the primary drug microparticle may be in the amorphous state, which can promote rapid dissolution of the drug once the seal coat is dissolved.

Generally, the primary drug microparticle can have a maximum diameter of no more than 50 μm such as, for example, no more than 25 μm, no more than 20 μm, no more than 15 μm, no more than 10 μm, no more than 9 μm, no more than 8 μm, no more than 7 μm, no more than 6 μm, no more than 5 μm, no more than 4 μm, no more than 3 μm, or no more than 2 μm. As used herein, the term “diameter” refers to greatest dimension of the primary drug particle.

The primary drug microparticle can have a minimum diameter of at least 100 nm such as, for example, at least 200 nm, at least 250 nm, at least 500 nm, at least 750 nm, at least 1 μm, at least 2 μm, at least 3 μm, at least 4 μm, or at least 5 μm.

The diameter of the primary drug microparticle can be characterized by a range having endpoints defined by any minimum diameter listed above and any maximum diameter identified above that is greater than the selected minimum diameter. For example, in some embodiments, the primary drug microparticle can have a diameter of from 200 nm to 25 μm, from 1 μm to 10 μm, from 2 μm to 10 μm, from 500 nm to 10 μm, from 1 μm to 15 μm, from 500 nm to 5 μm, or from 5 μm to 10 μm. In certain embodiments, the primary drug microparticle can have a diameter equal to any minimum diameter or any maximum diameter listed above. Thus, for example, the primary drug particle can have a diameter of 500 nm, 1 μm, 5 μm, 10 μm, 15 μm, etc.

The second step of the two-step process includes applying a seal coat to the primary drug microparticle. The seal coat surrounds the primary drug microparticle to interfere with interactions between drug on the surface of the primary drug microparticle and taste buds (FIG. 1B). In some embodiments, the seal coat completely and continuously surrounds the primary drug particle. In other embodiments, the seal coat may be discontinuous. The degree to which the seal coat is continuous or discontinuous may be designed according to the taste characteristics of the drug or drugs. The thickness of the coating may vary and will depend on the properties of the drug and the coating system used.

In some embodiments, the seal coat can include one or more pharmaceutically acceptable polymers that are insoluble in saliva but readily dissolve in an acidic environment such as the stomach. The pharmaceutically acceptable polymer may be any suitable homopolymer or copolymer.

In some embodiments, the seal coat can include commercially available pharmaceutical polymers such as, for example, a reverse enteric polymer. Reverse enteric polymers are insoluble at a pH greater than (more basic than) about pH 6.0, but are soluble in more acidic conditions. Consequently, reverse enteric polymers will not dissolve in saliva (pH 6.8-7.2) but will dissolve in the stomach (pH 1-4), thereby releasing the drug in the gastric environment for absorption and therapeutic effect. Thus, while the seal coat interferes with interactions between the drug and taste buds, the seal coat does not interfere with dissolution of the drug and subsequent drug absorption. In some embodiments, the seal coat can include only aqueous-based polymer systems so that issues related to organic solvents, including potential health risks from residual solvents and environmental pollution, can be avoided.

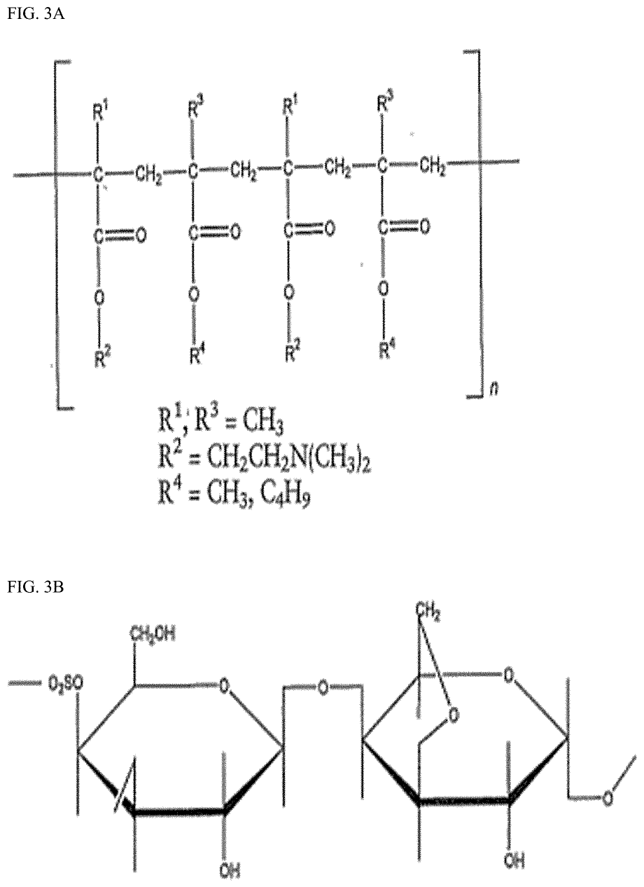

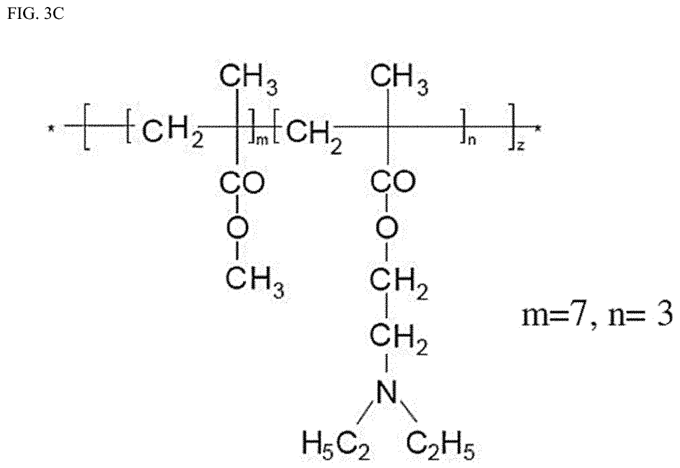

Exemplary commercially available pharmaceutically acceptable reverse enteric polymers include, but are not limited to, poly(butylmethylmethacrylate-co-2-dimethylaminoethyl) methacrylate-co-methyl methacrylate (FIG. 3A, also known as basic butylated methacrylate copolymer, e.g., EUDRAGIT E, Evonik Industries, Essen, Germany) and KOLLICOAT Smartseals (BASF, Ludwigshafen, Germany; FIG. 3C). KOLLICOAT Smartseal 30 D is a co-polymer that includes methyl methacrylate (MMA) and diethylaminoethyl methacrylate (DEAEMA) in a ratio of 6:4. It also contains macrogol cetostearylether 20 (˜0.6%) and sodium lauryl sulfate (˜0.8%) as stabilizers and has a solids content of 30%.

Other pharmaceutically acceptable polymer systems that exhibit preferential solubility in the stomach include, for example carrageenan (FIG. 3B). Carrageenan also has potential as a bitter blocking agent and is also reported to increase viscosity. Water soluble polymers also may be used for the seal coating, provided that the coating is sufficient to interfere with the drug from dissolving while in the oral cavity. Examples of such water-soluble polymers include, but are not limited to, hypromellose (hydroxypropyl methylcellulose) and polyvinyl alcohol.

In some embodiments, the seal coating can include one or more additives such as, for example, an excipient or an active agent that possesses acceptable taste characteristics. Exemplary excipients include, but are not limited to, a flavorant, a plasticizer (e.g., to assist with forming a film coating), a pigment, or a colorant.

The sealed microparticles can be readily incorporated into a variety of oral dosage forms including, but not limited to, orally dissolving tablets and orodispersible films. The small size of the coated microparticles also can reduce the sensation of grittiness in the mouth.

In some embodiments, the seal coating can be designed to possess a desired degree of flexibility so that it does not fracture when compressed. For example, the coated microparticles may be formed into a compression tablet. In such embodiments, the seal coating should have sufficient flexibility that is does not fracture during compression. Alternatively, the sealed microparticles may be formed into a chewable dosage form (e.g., a chewable tablet). In such embodiments, the seal coating should possess sufficient flexibility that the coating does not fracture when the chewable dosage form is chewed.

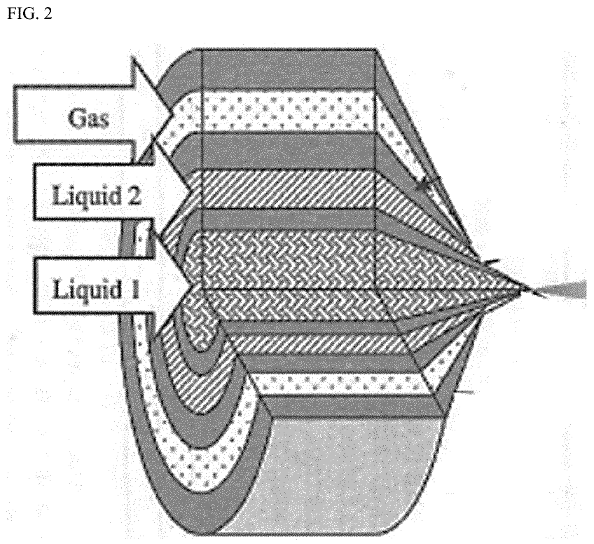

The seal coating may be applied to the primary drug microparticle using any suitable method known to those of ordinary skill in the art. In some embodiments, the seal coat may be applied using a three-layer concentric structured spray nozzle (FIG. 2). In other embodiments, the seal coating may be applied using microencapsulation techniques.

The nozzle design illustrated in FIG. 2 allows a solvent sprayed via the outer peripheral nozzle (Liquid 2 in FIG. 2) to coat another solvent sprayed via the inner center nozzle (Liquid 1 in FIG. 2) during the atomization process. Generally, the process includes a first step in which the primary drug microparticle is prepared. The process then includes a second step in which the seal coat is applied to the primary drug microparticle.

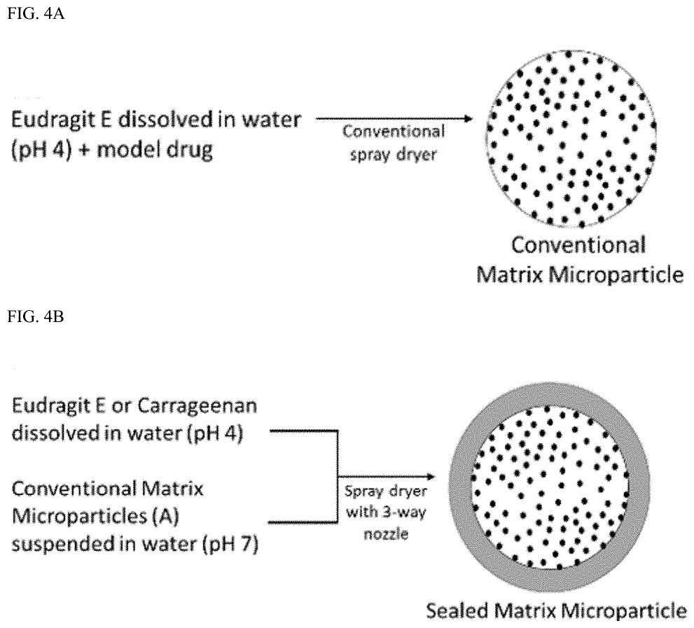

One exemplary process for preparing the coated microparticles described herein is illustrated in FIG. 4. FIG. 4A shows an exemplary method of preparing the primary drug microparticle. In this exemplary embodiment, the primary drug microparticle is prepared using conventional spray drying techniques (FIG. 4A). Any excipient known to one skilled in the art can be used to create the drug-containing primary microparticle. For example, EUDRAGIT E (Evonik Industries, Essen, Germany) may be used to form the primary drug microparticles since it is a well-known polymer used as a coating material for taste masking and can be spray dried. While illustrated schematically as a homogeneous mixture of a single drug within the matrix, the primary drug microparticle can include a single drug or any suitable combination of two or more drugs and/or two or more suitable excipients.

FIG. 4B illustrates an exemplary method for applying the seal coat to the primary drug microparticle. Using the three-way nozzle illustrated in FIG. 2, the primary drug microparticle is suspended in a suitable solvent as Liquid #1, the seal coat polymer is dissolved in water as Liquid #2. The primary drug microparticle and the seal coat polymer are spray dried using the three-way nozzle illustrated in FIG. 2, producing an exemplary continuously coated sealed matrix microparticle illustrated schematically in FIG. 4B. As explained above, the continuous coating shown in FIG. 4B is merely exemplary. In alternative embodiments, the coating may be discontinuous, provided the coating is sufficient to interfere with drug in the primary microparticle interacting with taste buds while in the oral cavity.

The suspension of primary drug microparticles may be prepared in an aqueous liquid or suitable organic solvent. The solvent in which the primary drug microparticle is suspended may be selected to minimize the likelihood and/or extent to which the matrix can dissolve and release drug from the matrix. Separately, the seal coat polymer can be dissolved in a suitable solvent, for example an aqueous solution of suitable acidity to dissolve the selected polymers (e.g., reverse enteric polymers). The two components may be fed into the three-way nozzle for spray drying by conventional means For example, the two components may be fed into the three-way nozzle from two different channels that do not mix until just before spraying at the tip of the nozzle. Thus, with appropriate selection of excipients, the spray drying process can be completely aqueous based. In such embodiments, the process eliminates any concerns related to the use of organic solvents, solvent recovery, and residual solvents in the microparticles.

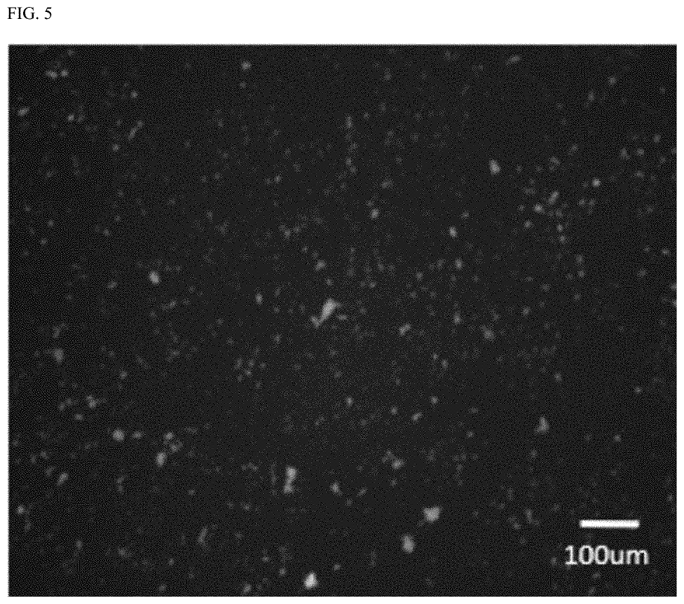

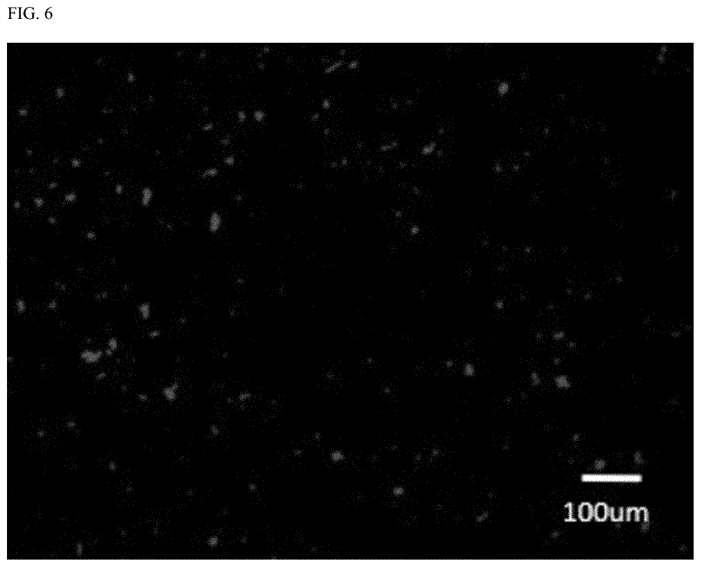

FIG. 5 shows matrix microparticles containing acetaminophen and EUDRAGIT E (Evonik Industries, Essen, Germany) in a 1:4 ratio prepared using conventional spray drying. Acetaminophen auto-fluoresces and appears as green in FIG. 5. This image clearly shows drug located on the exterior surface of the particles and confirms that conventional spray drying produces matrix microparticles. The microparticles from FIG. 5 as the primary drug particles for coating with a seal coat containing Rhodamine B according to the second spray-drying step illustrated in FIG. 4B. FIG. 6 shows the merged image of the red and green channels of these microparticles. The red fluorescence is from Rhodamine B in the seal coat and there is a distinct absence of green from the acetaminophen. Thus, the Rhodamine-containing coating covered the primary drug microparticles and formed a seal around the primary drug microparticle structures. Furthermore, there is a lack of yellow (colocalization of the red and green channels) in FIG. 6, suggesting that the drug does not partition from conventional matrix microparticles into the seal coating during the second spray-drying step.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### Examples

Spray drying was performed with the Buchi B-290 mini spray dryer (Buchi Corporation, Flawil, Switzerland). For the core solution, a two-fluid nozzle and the inbuilt peristaltic pump were used in delivery of the feed solution. The cores were coated using a three-fluid nozzle and an external peristaltic pump (MICROFLEX, Cole-Parmer, Vernon Hills, Ill.). The single peristaltic pump is equipped with two individual pump heads, allowing for synchronized feed from the individual solutions. Flexible tubing with an internal diameter of ⅛ inch and uniform length (50 inches) was used for each pump. The combined pump teed rate used for all spray drying was 4 mL/min±0.4 (the variation arises between the two different pump systems and following minor differences in calibration). The SD parameters included an inlet temperature of 110° C.±10 to achieve an outlet temperature of 49° C.±2, and 100% aspirator rate.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. If any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A pharmaceutical composition comprising:
a primary microparticle comprising:
a matrix; and
at least one drug dispersed within the matrix; and
a seal coating dispersed over at least a portion of the primary microparticle, the seal coating comprising a pharmaceutically acceptable polymer.

2. The pharmaceutical composition of claim 1, wherein the primary microparticle has a diameter of no more than 25 μm.

3. The pharmaceutical composition of claim 1, wherein the primary microparticle has a diameter of no more than 10 μm.

4. The pharmaceutical composition of claim 1, wherein the primary microparticle has a diameter of from about 1 μm to 10 μm.

5. The pharmaceutical composition of claim 1, wherein the seal coating comprises a reverse enteric coating.

6. The pharmaceutical composition of claim 5, wherein the reverse enteric coating comprises poly(butylmethylmethacrylate-co-2-dimethylaminoethyl) methacrylate-co-methyl methacrylate or a co-polymer of methyl methacrylate and diethylaminoethyl methacrylate in a ratio of 6:4.

7. The pharmaceutical composition of claim 1, wherein the seal coating comprises carrageenan, hypromellose, or polyvinyl alcohol.

8. The pharmaceutical composition of claim 1, wherein the primary microparticle comprises two or more drugs dispersed within the matrix.

9. A method for preparing a pharmaceutical composition, the method comprising:
obtaining a primary microparticle comprising:
a matrix, and
drug dispersed within the matrix; and
applying a seal coating to at least a portion of the primary microparticle, the seal coating comprising a pharmaceutically acceptable polymer.

10. The method of claim 9, wherein the primary microparticle comprises at least two drugs dispersed within the matrix.

11. The method of claim 9, wherein the seal coating comprises a reverse enteric coating.

12. The method of claim 11, wherein the reverse enteric coating comprises poly(butylmethylmethacrylate-co-2-dimethylaminoethyl) methacrylate-co-methyl methacrylate or a co-polymer of methyl methacrylate and diethylaminoethyl methacrylate in a ratio of 6:4.

13. The method of claim 9, wherein the seal coating comprises carrageenan, hypromellose, or polyvinyl alcohol

14. The method of claim 9, wherein the seal coating is continuous.

15. The method of claim 9, wherein the seal coating is discontinuous.

16. The method of claim 9, wherein obtaining the primary microparticle comprises spray drying drug and a matrix composition.

17. The method of claim 16, wherein the primary microparticle has a diameter of no more than 25 μm.

18. The method of claim 17, wherein the primary microparticle has a diameter of from 1 μm to 10 μm.

19. The method of claim 9, wherein applying the seal coating to the particles comprises:
suspending a plurality of the primary microparticles in a suitable solvent;
dissolving the pharmaceutically acceptable polymer in an appropriate solvent; and
spray drying the plurality of primary microparticles with the pharmaceutically acceptable polymer.

20. The method of claim 19, wherein the spray drying occurs through a three-way spray drying nozzle, the three-way spray drying nozzle comprising:
a first liquid channel;
a second liquid channel; and
a gas channel.

21. The method of claim 20, wherein the suspended primary microparticles are fed through the first liquid channel and the dissolved pharmaceutically acceptable polymer is fed through the second channel.

22. The method of claim 9, wherein applying the seal coating to the particles comprises:
suspending a plurality of the primary microparticles in an aqueous medium;
dissolving a reverse enteric polymer in an aqueous medium having a pH sufficiently acidic to dissolve the reverse enteric polymer; and
spray drying the plurality of primary microparticles with the dissolved reverse enteric polymer.
